(19) United States
(12) Patent Application Publication
(10) Pub. No.: US 2003/0153841 A1
(43) Pub. Date: Aug. 14, 2003

(54) Method for investigating neurological function

(76) Inventor: Kerry Kilborn, Renfrewshire (GB)

Correspondence Address: Fleshner & Kim, PO Box 221200, Chantilly, VA, 20153-1200, US

(21) Appl. No.: 10/204,191

(22) PCT Filed: Feb. 19, 2001
(86) PCT No.: PCT/GB01/00697

(30) Foreign Application Priority Data
Feb. 19, 2000 (GB) 0003853.9

(51) Int. Cl. A61B 5/04

(52) U.S. Cl. 600/544

(57) ABSTRACT

A method for investigating neurological function, especially parameters in neurological function that are associated with disease. The method uses electroencephalographic (EEG) potentials generated by the workings of the brain combined with cognitive activation procedures or tasks to allow the investigation of neurological functions of the brain. The collection of brain electrical signals in conjunction with the subjects carrying out one or several cognitive tasks allows the responses to be compared either to the results of other subjects or to results obtained from the same subjects under different conditions.

The present application relates to a method for investigating neurological function, especially parameters of neurological function that are associated with disease. Similar implementations of the method may be created for different neurological diseases which are amenable to useful clinical assessment by this method. It relates in particular to a method of investigating neurological function which incorporates (a) High channel count EEG; (b) cognitive activation procedures (“tasks”); (c) a pre-constructed normative database based on specific cognitive activation procedures carried out with relevant healthy and/or clinical populations; and (d) software-based computational procedures which use an individual's test data together with the relevant database to provide information such as classification and severity measures as an aid to clinical diagnosis.

The key innovative aspect of this invention involves the combined use of cognitive tasks with EEG for the purpose of diagnosing central nervous system disorders in individuals. EEG his a long history of clinical use, but the method proposed in this application is fundamentally different to existing clinical uses (cf. La Rue, A. (1992), Aging and Neuropsychological Assessment, Plenum Press: New York, page 46). For individual clinical diagnosis of neurological disorders, EEG alone is used but not combined with cognitive tasks. A particular form of EEG called “evoked potentials” is used to assess sensory function (Brainstem Auditory Evoked Potentials, Visual Evoked Potentials), but not cognitive function. Cognitive tasks are used in some diagnostic applications, but not combined with EEG. Current clinical practice does not include the application of cognitive tasks combined with EEG for diagnosis of neurological disorders, in fact the abovementioned references show that current knowledge teaches away from the ideas disclosed herein.

Common neurological health problems include the Primary Degenerative Dementias (e.g., Alzheimer's disease, Lewy Body Dementia, Vascular Dementia), Affective Disorders (e.g., Depressive Disorder), Parkinson's disease, stroke, schizophrenia, multiple sclerosis, addictive disorders, dyslexia, autism, and attention deficit disorders.

Each of these present problems in diagnosis, monitoring, and optimising therapy. Tools to assist in these efforts are often non-existent, unavailable, or of very limited value.

As an illustration, the principal symptoms of both Alzheimer's disease and depression are degradation of memory and concentration. A definitive diagnosis often proves very difficult to achieve based solely on clinical presentation, and there are as yet no known biological or radiological investigations that can reliably determine the nature of the primary disorder. The existence of other forms of dementia (e.g., vascular dementia) with similar presentations further complicates the diagnostic picture.

Better diagnostic tools are becoming increasingly important as the range of treatment options for neurological disorders increases. For example, effective treatments for affective disorders (e.g., antidepressant medication) have been available for many years, but very recently several new options for treating Alzheimer's Disease have become available. Though not yet available for clinical use, the advent of general neuroprotective agents offer the possibility that previously untreatable disorders can be effectively treated, provided they can be clearly diagnosed at early enough stages. One goal of clinical practice has been to ensure that patients always receive the available treatment. To meet this goal, methods are required which will increase diagnostic accuracy and help to reduce the risk of inappropriate therapies.

Alzheimer's Disease is a progressive degenerative disease of the brain and is the most common form of dementia, affecting more than 10% of people over the age of 65. As well as the human cost of Alzheimer's Disease, the financial implications associated with this pathology exceed $100 million annually in the US alone at the present time. Furthermore, the incidence of Alzheimer's Disease is predicted to increase in the future because the population is ageing.

A major problem in the treatment of Alzheimer's Disease at the present time is that there exists no conclusive diagnostic test which can be used to confirm that the patient has Alzheimer's Disease. Currently, the only definitive diagnosis that can be made is by post mortem examination. Due to the lack of a conclusive test it is difficult to know which people really have the disease. It is therefore difficult to know who should be treated.

If it were possible to detect Alzheimer's Disease earlier in its progression then treatment could be improved. Furthermore, it would be easier to exclude Alzheimer's Disease as a possible cause of symptoms in unaffected patients.

The value of an early stage diagnostic tool for Alzheimer's Disease is shown in a report issued by The Ronald and Nancy Reagan Institute, which has as its top priority the aim of accelerating the discovery of treatments that can intervene in the progression of Alzheimer's Disease before symptoms appear. The Reagan Institute predicts that a five year delay at the onset of symptoms could cut the number of people inflicted by half, saving $50 billion annually in the United States.

Therefore, there is an exceptional demand for tools for use in diagnosis of Alzheimer's Disease and other neurological and psychological disorders. Although this has been an aim of medical research for decades, there remains at the present time an important unmet need for tests suitable for use in diagnosis.

As mentioned above, the only current method of providing a definitive diagnosis of Aizheimer's Disease can be carried out only after the death of a patient. Current clinical practice in Alzheimer's Disease diagnosis relies mainly on a range of paper-and-pencil tests, and occasional anatomical imaging, administered by a variety of health professionals including neuropsychologists, psychiatrists, radiologists, and others. Rather than identifying characteristic markers of Alzheimer's Disease, current clinical tests aim to exclude other possible diagnoses.

The state of the art in detection of Alzheimer's Disease, particularly through paper-and-pencil type tests is summarised in the paper “The nature and staging of attention dysfunction in early (minimal and mild) Alzheimer's Disease: relationship to episodic and semantic memory impairment”. (Richard J Perry et al., Neuropsychologia 38 (2000) 252-271). This paper, written by an authority in the field, discusses a variety of neuropyschological tests concluding that one of the most successful is the so called Stroop test in which subjects read out words, red, green, blue, tan that are printed in ink of an incongress colour e.g. the word red is printed in blue. Patients then read another list of colour names, this time they name the colour of ink in which the word is printed instead of the actual word that is written. However, the best pencil-and-paper tests known miss many early-stage Alzheimer's Disease symptoms, and frequently cannot reliably distinguish Alzheimer's Disease from other diseases.

Many clinicians claim diagnostic success using interviews and medical histories of a patient. These are known to be up to 85′?, correct for patients with advanced disease who do not have other diseases and are not taking medications; unfortunately the elderly often have other diseases which confuse diagnosis. The practical level of confidence is usually in the order of 75%.

The disadvantages of this subjective approach are, firstly, that the results cannot be fully trusted. A 75% confidence level leads to the risk of missing an unacceptable number of patients and of unnecessarily worrying and treating some patients who do not in fact have the disease. Secondly, subjective approaches can only be carried out successfully once the disease is already highly advanced. A third disadvantage of the subjective techniques is that they can only be carried out by a skilled clinician whose time is expensive. Several interviews of, say, one hour each are required.

A supplementary approach to diagnosis is brain scanning. For example, CAT and SPECT scans have been used to monitor changes in brain blood flow which are thought to be implicated in the development of Alzheimer's Disease. These scans have proved able to add confidence of the diagnosis of Alzheimer's Disease, but the scanning techniques are not without their drawbacks. The cost of the scanning equipment is high, and repeat testing increases the danger of exposure to radioactive isotopes. Furthermore, the scanning techniques have mainly been tried out in difficult to diagnose patients who already have advanced symptoms and have not been proved efficacious for early stage patients

Another method that has been used in diagnosis of Alzheimer's Disease is genetic screening. However, there is no single gene for Alzheimer's Disease and as Alzheimer's Disease is common amongst the ageing population, having an elderly relative with Alzheimer's Disease is not fully predictive of a familial link.

Variations in the genes ApoE 2, ApoE 3 and ApoE 4 are thought to influence Alzheimer's Disease risk. However, genetic testing can only show who is at risk, not whether the disease has started in a particular individual. Widespread screening is unlikely to positively influence the outcome of Alzheimer's Disease progression. Genetic testing is certainly not an early onset diagnostic technique.

The aim of the present invention is to provide a method of assessing aspects of brain function known to be linked in some circumstances to neurological and psychological disorders and thereby to provide information which can be used by a clinician in making a diagnosis. Ideally, the invention aims to provide an early onset test which can be applied accurately whilst the disease is still in its early stages allowing treatment to be begun. The invention aims also to provide a test which is economic and which can be carried out quickly and easily by technicians rather than requiring the time of highly trained clinicians. The test can be applied repeatedly to an individual, and so can serve not only as a diagnostic tool but also as an aid to ongoing management of therapy.

Although the use of this technique for detecting Alzheimer's Disease is the primary focus of this example description, the technique will also be applied to other aspects of brain function, particularly diseases such as Parkinson's disease, depression, stroke, schizophrenia, multiple sclerosis, addictive disorders, dyslexia, autism, and attention deficit disorders. Detection of these and other disorders which have disruption or change to higher cognitive functions among their primary symptoms is an additional aim of the invention herein disclosed.

According to a first aspect of the present invention there is provided a method of characterising an aspect of the neurological function of a subject comprising the steps of:

(a) providing stimuli to a subject, said stimuli being chosen to cause the subject to carry out a particular neurological act;

(b) monitoring the electroencephalographic (EEG) response of the brain whilst said neurological act is carried out; and

(c) comparing said response of the brain with a database of normative responses in trial subjects, said database also containing information about an aspect of neurological health of those trial subjects.

(d) the use of statistical methods to carry out said comparison which are appropriate to the task of characterising an individual's test data as an aid to clinical diagnosis and management of therapy.

According to a second aspect of the present invention there is provided a method of characterising an aspect of the neurological function of a subject comprising the steps of:

(a) providing stimuli to a subject, said stimuli being chosen to cause the subject to carry out a particular neurological act;

(b) monitoring the electroencephalogic (EEG) response of the brain whilst said neurological act is carried out; and

(c) comparing said response of the brain in a subject or group of subjects, where the subjects are, or subject is, at different stages of treatment with a therapeutic, which includes pre- and post-treatment; and

(d) using statistical methods to carry out said comparison, which are appropriate to the task of characterising test data as an aid to commercial research and testing of therapetics.

Preferably, the aspect of neurological function of a subject which is characterised is a parameter associated with the presence or absence of a neurological disease.

More preferably, the neurological disease is one which is known or suspected to cause changes to higher cognitive function such as memory, attention and language.

Preferably, the EEG potentials are measured by a sensor array applied to the subject's head.

Most preferably, at least a 128 channel sensor array is used to measure EEG potentials on the surface of a subject's head.

Preferably, the stimuli are auditory, visual, and/or tactile.

Typically, the subject will be required to carry out actions in response to stimuli.

The aspect of neurological function may be memory and the stimuli may preferably be repetitive presentation of information, with the subjects being required to make a response indicating whether they recognise information presented to have been repeated.

The aspect of neurological function may be response inhibition and the stimuli may preferably be the presentation of number words in one of a plurality of colours superimposed over bars, with the subject being required to respond to the word or the bar depending on the colour in which the number word is presented.

The aspect of neurological function may be the ability to dynamically change a response selection rule in a choice task and the stimuli may preferably be two different visual images, with the subject being required to make one response to one visual image and a second response to a second visual image, wherein the subject is periodically required to swap the responses made to the visual stimuli.

The aspect of neurological function may be interhemispheric transfer and the stimuli may preferably be visual or auditory stimuli presented to either the left or right visual field or ear of the subject wherein the subject is required to make a response to indicate perception of the stimuli, wherein the specific peak latency of the left and right hemispheres of the brain are separately measured and the difference between these times is calculated.

The aspect of neurological function may be language comprehension and/or production ability and the stimuli may preferably be visual or auditory language or other symbolic representations, with the subject being required to make responses either verbally or manually which indicate operation of a particular language function, especially those subject to selective impairment by neurological disease or damage.

The response of the brain to more than one set of stimuli may be measured.

The responses of the brain to more than one different set of stimuli may, in an otherwise known method, be taken into account simultaneously and compared with the database of responses in trial subjects.

The invention will now be described by way of example only with reference to the following Figure in which:

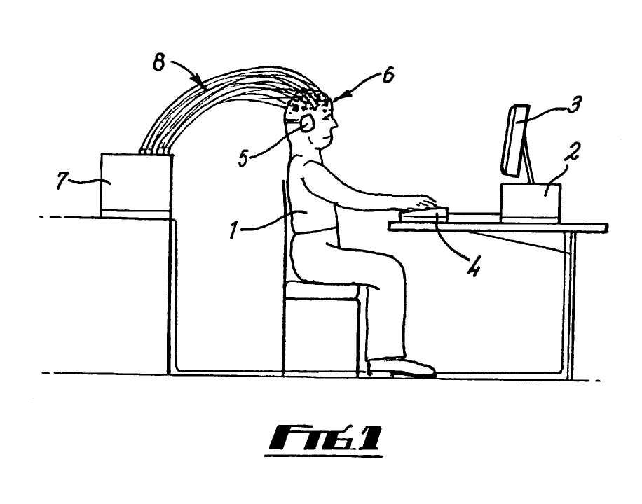

FIG. 1 shows in perspective view the key apparatus used in the present invention.

The subject of this application is a new method for measuring aspects of neurological function. In particular, the following example describes the invention being applied to the detection of a disease which primarily affects elderly people.

The test is composed of one or several cognitive tasks which are employed in combination with the collection of brain electrical signals. Essentially, the patient's brain is driven by the tests to carry out specific neurological functions. Multiple measurements of brain electrical signals and behavioural responses are then collected, analysed by statistical methods, compared against a database of results from diseased and normal patients and used to provide an index value which can be used by a clinician. Furthermore, results from multiple tests and multiple measures from individual tests may be analysed together, providing further indices of greater accuracy and disease specificity.

Important innovative elements of this test are the specific designs of the cognitive tasks and the use of time-locked EEG (evoked potentials) to measure the brain's response during task performance.

Referring to FIG. 1, a patient 1 sits in front of a test controlling computer 2 which has a VDU 3, input keys 4 and audio speakers 5. A dense-array EEG system 6 is affixed to their head to measure EEG potentials across the head surface as time series. In the present example, a commercial (128-channel) digital dense-array EEG system is used.

The tasks in the diagnostic tool are designed to tap into features of perceptual and higher cognitive function that are known to deteriorate relatively early in the disease process. These include memory, attention, language, and certain vision and audition processes. More specifically, the VDU 3 and headphones 5 provide instructions to the patient to perform a battery of tasks designed to tap into perceptual and higher cognitive functions including memory, attention and language. Measurements of brain electrical function are collected during task performance by means of the EEG system 6.

EEG responses are measured by a data processing computer 7, connected to the sensor array 6 by a plurality of wires 8. The data processing computer 7 evaluates the potentially pathological patterns of brain activity in a clinical subject by comparing them against a database of appropriately normed data from healthy and known pathological samples. By objectively comparing resultant data with appropriate populations norms, indicators pointing to an evaluation of the presence and graded severity of a pathological brain state are calculated.

Database

Elderly healthy subjects volunteer to participate in test sessions. Possible patients are then compared against the baseline provided by the elderly (i.e., age-matched) healthy subjects. This provides a measure of the decline in cognitive brain performance due to the neurological disorder, over and above the effects of normal ageing. In practice, clinicians will use the test battery to obtain a data set from a candidate patient and then use this information in forming their diagnosis. The results will then be compared against an appropriate subset of the normative database. Objective measures of deviation can be quantified and charted, providing the clinician a concise summary of key markers characteristic of the neurological disorder versus normal performance.

Apparatus

The commercial EEG systems 6 are supplied by Electrical Geodesics Inc (Eugene, Oreg., USA). The system consists of an amplifier, several sizes of electrode nets, control computers and control software. A custom user interface shell to enslave the EGI software is provided in the invention herein disclosed.

Data outputs from the battery of tests may be used to prepare parameters based on the results of individual tests. However, it is known that individual changes in brain function may be caused by more than one pathology. This is why more than one test may be required in diagnosis of some neurological pathologies.

In a further embodiment of the present invention it is recognised that results from more than one of the tests may be fed into known mathematical processing techniques to provide measurements correlated with diagnoses of diseases which are more specific to individual pathologies than results of individual tests.

In a yet further embodiment of the present invention, the imaging capability of the geodesic EEG sensor array is utilised further. Aspects of neurological function are often localised to individual parts of the brain. This can be extended by providing tests which drive individual areas of the brain to carry out tasks and by then measuring parameters of the EEG response of those individual areas of the brain.

The tests described herein will be useful for clinicians who are responsible for diagnosis and management of neurological disorders which affect higher cognitive function and for pharmaceutical companies requiring sensitive tests of drug action aimed at neurological disorders which affect higher cognitive function. In the latter case, group comparisons may be preferred to individual diagnoses.

Throughout this application, unless the context requires otherwise, the word “comprise” or variations such as “comprises” or “comprising” will be understood to imply the inclusion of a stated integer or group of integers but not the exclusion of any other integer or group of integers. Also the term “neurological” in this document is meant to encompass both neurological and psychiatric functions.

Further improvements and modifications clear to one skilled in the art may be made within the scope of the invention herein described.

1. A method of characterising an aspect of the neurological function of the subject comprising the steps of:
(a) providing stimuli to a subject, said stimuli being chosen to cause the subject to carry out a particular neurological act; and
(b) monitoring the electroencephalographic response (EEG) of the brain whilst said neurological act is carried out.

2. A method of characterising an aspect of the neurological function of a subject, as in claim 1, comprising the additional steps of:
(c) comparing said response of the brain with a database of normative responses in trial subjects, said database also containing information about an aspect of neurological health of these trial subjects; and
(d) using statistical methods, to carry out said comparison, which are appropriate to the task of characterising an individuals test data as an aid to clinical diagnosis and management of therapy.

3. A method of characterising an aspect of the neurological function of the subject comprising the additional steps of:
(c) comparing said response of the brain in a subject or group of subjects, where the subject is, or the subjects are, at different stages of treatment with a therapeutic, which includes pre and post treatment; and
(d) using statistical methods, to carry out said comparison, which are appropriate to the task of characterising test data as an did to commercial research and testing of therapeutics.

4. A method of characterising an aspect of the neurological function of a subject, as in any of the previous claims, wherein the aspect of neurological function of the subject which is characterised is a parameter associated with the presence or absence of a neurological disease.

5. A method of characterising an aspect of the neurological function of a subject, as in claim 4, wherein the neurological disease is one which is known or suspected to cause changes to higher cognitive functions, such as memory, attention and language.

6. A method of characterising an aspect of the neurological function of a subject, as in any of the previous claims, wherein the EEG potentials are measured by a sensor array applied to the subject head.

7. A method of characterising an aspect of the neurological function of a subject, as in claim 6, wherein the multi-channel sensor array is at least a 128 channel sensor array.

8. A method of characterising an aspect of the neurological function of a subject, as in any of the previous claims, wherein the stimuli are auditory, visual and/or tactile.

9. A method of characterising an aspect of the neurological function of a subject, wherein the subject will be required to carry out actions in response to stimuli.
